(19) United States
(12) Patent Application Publication
(10) Pub. No.: US 2006/0167548 A1
(43) Pub. Date: Jul. 27, 2006

(54) Non-linear spinal fusion interbody spacer

(76) Inventor: Roger P. Jackson, Prairie Village, KS (US)

Correspondence Address: LAW OFFICE OF JOHN C. MCMAHON, P.O. BOX 30069, KANSAS CITY, MO, 64112, US

(21) Appl. No.: 11/384,799

(22) Filed: Mar. 20, 2006

Related U.S. Application Data
(63) Continuation-in-part of application No. 10/842,295, filed on May 10, 2004.
Application No. 10/842,295 is a continuation of application No. 10/649,412, filed on Aug. 27, 2003.
Application No. 10/842,295 is a continuation-in-part of application No. 09/644,722, filed on Aug. 23, 2000, now Pat. No. 6,666,888.
Application No. 10/842,295 is a continuation-in-part of application No. 10/651,800, filed on Aug. 29, 2003.

(51) Int. Cl. A61F 2/44

(52) U.S. Cl. 623/17.11

(57) ABSTRACT

An interbody device includes a solid interior and a non-linear body with opposed top and bottom abutment surfaces that are asymmetrically convex, the device being sized and shaped to be operably positioned between a pair of opposing vertebrae for support and/or fusion. The non-linear body has opposed first and second sides, the first side exhibiting a substantially convex profile and the second side exhibiting a substantially concave profile when viewed from the top or bottom, resulting in a arc- or kidney-shaped configuration. Furthermore, both sides include surfaces that are concave running from the top to the bottom and further include channels. The top and bottom surfaces may include ridges or teeth for facilitating positioning, attachment and fusion to bone.

### CROSS REFERENCE TO RELATED APPLICATIONS

The present application is a continuation in part of pending U.S. patent application Ser. No. 10/842,295 filed May 10, 2004, which is a continuation in part of U.S. patent application Ser. No. 10/649,412 filed Aug. 27, 2003 and a continuation in part of U.S. patent application Ser. No. 09/644,722 filed Aug. 23, 2000, now U.S. Pat. No. 6,666,888 and a continuation in part of U.S. patent application Ser. No. 10/651,800, filed Aug. 29, 2003, all of which are incorporated by reference herein.

### BACKGROUND OF THE INVENTION

The present application is directed to an interbody device for implantation between a pair of adjacent vertebrae for the purpose of providing support to and promoting fusion between the vertebrae, and more particularly, to an intervertebral implant device having a non-linear design.

In the human spine, the pad or disc between vertebrae can become damaged and deteriorate due to injury, disease or other disorders. Upon such an occurrence, the discs may narrow or flatten, resulting in painful mechanical instability that may ultimately progress to complete disc failure with associated disc space collapse. In an attempt to remedy such narrowing, flattening and ultimate failure, various procedures are employed that typically entail removal of the faulty disc and strategic placement of bone chips and/or mechanical implants between the vertebrae for the purpose of providing support and maintaining disc space height and lordotic configuration of the vertebrae.

In addition to providing support and lordotic alignment, an underlying objective of such mechanical implants is to promote fusion between adjacent vertebrae. Thus, such implants are often referred to as fusion cage or intervertebral fusion devices or spacers. Implants of this nature typically consist of a hollow central cavity with apertures that can be packed with bone so as to promote bone growth and fusion between the implant and the surrounding bone. Specifically, such apertures provide means for the bone to communicate through the implant, thus promoting arthrodesis or fusion. In some procedures, multiple interbody devices are used together and bone fusion material is packed between a pair of devices that are placed in close proximity to one another and extend between the vertebrae to promote growth of bone and fusion between the vertebrae. Over a period of time, the body encompasses the implant and locks it into place resulting in a strong vertebral column.

While the promotion of bone growth to better incorporate the implant into the body is vital, designing implants with apertures and hollow cores significantly reduces the structural integrity of such an implant, especially when made of non-metallic materials. The body's natural forces, which are aided by gravity, subjects intervertebral implants to significant compression forces. In addition to these forces, implants may be damaged due to impact from sports and other inadvertent collisions.

Thus, it is desirable to provide implants having a high compression strength resulting in an implant with a longer life span. Cage fusion implants and other implants utilizing apertures and hollow cores are problematic due to characteristically low compression strengths and/or brittleness that are adverse to the implant's life span.

It is also desirable that such devices engage as much bone surface as possible to provide support to the bone and to reduce the likelihood of subsidence of the device into the bone, resulting from contact pressure of the interbody device or spacer against an intervertebral surface of a vertebra. Subsidence can occur since part of the bone is somewhat spongy in nature, especially near center regions of the opposing intervertebral surfaces.

Still further, it is desirable to provide implants that promote stability of the implant device by promoting bone growth or fusion thereabout and can be installed with a minimal amount of cutting into and reshaping of the vertebral bones to only an extent necessary to correct the structure and function of the spine. Thus, it is desirable to conform an interbody spacer to the shape of the vertebral surfaces of adjacent vertebrae, which surfaces are shallowly concave, rather than conform the vertebrae to the shape of the interbody spacer.

### SUMMARY OF THE INVENTION

An interbody or intervertebral spacer device for placement between a pair of adjacent vertebrae that facilitates fusion of adjacent bone structures in addition to providing a strong implant includes a non-linear body substantially defined by a pair of opposed abutment surfaces, a first side surface having at least a curved portion and a second side surface disposed substantially opposite the first surface. The spacer body is substantially non-linear in a direction running along and between the first and second sides. The spacer device may include a kidney, cashew, peanut, lunar or crescent shaped body or body portion with the first surface having a convex profile and the second substantially oppositely facing surface having a concave profile. The first surface may be sized and shaped to be placed near an outer edge or periphery of a vertebra, at an anterior or lateral region of the vertebra. When implanted, the second surface faces toward an interior of the intervertebral space. The non-linear, curvate or arcuate shape of the spacer allows for advantageous positioning thereof near a periphery of each of the vertebrae, and thus in a more stable location between the vertebrae as compared to central or inner regions where the bone is more spongy in nature and thus where undesirable subsidence of the device into the bone is more likely to occur.

At least one, and typically both of the first and second surfaces are concave running between the abutment or top and bottom surfaces. The device has a fixed shape that may continuously increase in thickness or height from back to front, the thickness measured between the abutment surfaces, such that when implanted, a portion of the device nearer an anterior region of the vertebrae is thicker or taller than near a posterior region thereof. Such thickness may be in the form of a curved raised portion or arcuate ridge on each abutment surface that is spaced from an anterior surface thereof so as to better conform to a curvature of the adjacent vertebrae.

The device has a compact design with a solid interior and may in some embodiments further include grooves or channels running between the top and bottom surfaces and partially through the first and second surfaces, such grooves for accommodating bone-growth and facilitating fusion of the adjacent vertebrae. Furthermore, top and bottom surfaces may have ridges, knurling or teeth and/or be slanted to engage adjacent bone to provide secure placement between the vertebrae.

### OBJECTS AND ADVANTAGES OF THE INVENTION

Therefore, it is an object of the present invention to overcome one or more of the problems with interbody spacers described above. Further objects of the present invention are: to provide an interbody spacer device with a non-linear elongate body; to provide such a device with a solid interior; to provide such a device having a shape that follows a curvature of adjacent vertebrae; to provide such a device that is sized and shaped to limit or reduce subsidence of the device into bone; to provide such a device with grooves or channels on surfaces thereof for promoting fusion between the vertebrae; to provide such a device having one or more concave surfaces that follow the curvature of the adjacent vertebrae; to provide such a device having convex upper and lower vertebrae abutment surfaces; to provide such a device having teeth or ridges on upper and lower abutment surfaces thereof in order to better engage adjacent vertebrae and maintain position with such teeth; to provide such a device having a solid interior cavity to provide exceptionally strong structural integrity; to provide such a device with sufficient compression strength to ensure a long life span; to provide such a device having a compact structure with a reduced volume and weight; to provide such a device designed to promote ease of installation; to provide such a device that is capable of installation without the use of screw-torque or other screw-torque yielding installation devices that may bite into or otherwise degrade the surface of adjacent bone; and to provide such a device that is relatively easy to construct, inexpensive to produce and especially well-suited for the intended usage thereof.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an enlarged perspective view of an interbody spacer of the invention.

FIG. 2 is a top plan view of the spacer of FIG. 1.

FIG. 3 is a side elevational view of the spacer of FIG. 1.

FIG. 4 is a front elevational view of the spacer of FIG. 1.

FIG. 5 is a rear elevational view of the spacer of FIG. 1.

FIG. 6 is a cross-sectional view taken along the line 6-6 of FIG. 4.

FIG. 7 is a partial side elevational view illustrating the spacer of FIG. 1 disposed between vertebrae with opposing top and bottom spacer surfaces, shown in phantom, the top and bottom surfaces being sloped to reflect a curvature of the cooperating top and bottom vertebral surfaces.

FIG. 8 is a partial front elevational view of the spacer and vertebrae of FIG. 7, with the top and bottom spacer surfaces shown in phantom.

FIG. 9 is a partial top plan view of the spacer (shown in phantom) and one vertebra of FIGS. 7 and 8, illustrating an ideal placement of the spacer relative to anterior and posterior regions of the vertebra.

FIG. 10 is an enlarged perspective view of an alternative embodiment of an interbody spacer according to the invention shown in a first orientation.

FIG. 11 is a bottom plan view of the spacer of FIG. 10.

FIG. 12 is a rear elevational view of the spacer of FIG. 10 shown in a second inverted orientation.

FIG. 13 is a side elevational view of the inverted spacer of FIG. 12.

FIG. 14 is a side elevational view of the inverted spacer of FIG. 12 opposite the side shown in FIG. 13.

FIG. 15 is a cross-sectional view taken along the line 15-15 of FIG. 10.

FIG. 16 is a partial side elevational view of the spacer of FIG. 10 showing the spacer disposed between vertebrae.

FIG. 17 is a partial front elevational view of the spacer and vertebrae of FIG. 16 also showing a second identical spacer in front elevation and inverted, illustrating an ideal placement of the pair of spacers relative to the vertebrae.

FIG. 18 is a partial top plan view of the spacers and one vertebra of FIG. 17.

FIG. 19 is a partial fragmentary side elevational view similar to FIG. 16, with portions broken away to show the detail thereof.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

It is also noted that any reference to the words top, bottom, up and down, and the like, in this application refers to the alignment shown in the various drawings, as well as the normal connotations applied to such devices, and is not intended to restrict positioning of the spacers in actual use. It is also noted that reference to words such as front, back, anterior and posterior used in this application also refer to the alignment shown in the various drawings, and in particular, when possible, with reference to the human spine and human body, but also is not intended to restrict positioning of the spacers in actual use.

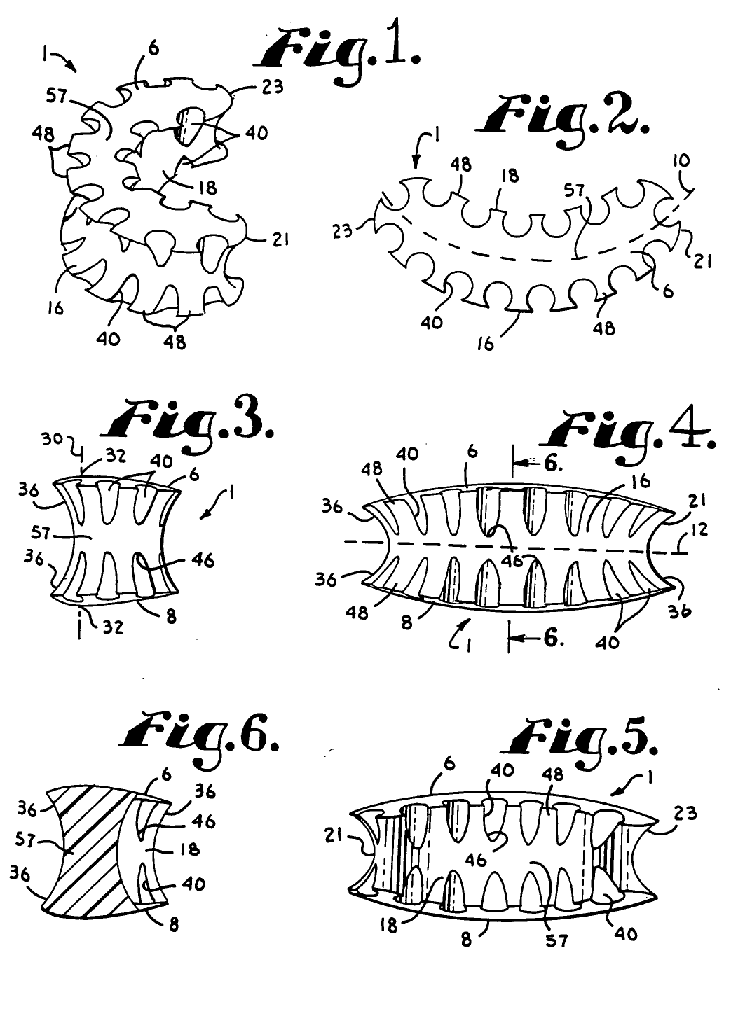

With reference to FIGS. 1-9, the reference numeral 1 generally designates a spinal fusion interbody spacer device of the present invention. The device 1 is used to maintain proper spacing between a pair of adjacent vertebrae 3 and 4 of a human spine 5 as a replacement for the intervertebral disc and to promote fusion between the vertebrae 3 and 4, preferably in conjunction with other implants, as will be described more fully below. The device 1 is preferably constructed from a single, unitary and rigid blank or molded structure. In particular, the device 1 has a superior or upper abutment surface 6 and an inferior or lower abutment surface 8. The device 1 is elongate, having opposed first and second, or front and back, outwardly facing surfaces 16 and 18, respectively. When viewed from the top as illustrated in FIG. 2, the device is non-linear, having a front, first or anterior surface 16 with a substantially convex outer profile while the back, second or posterior surface 18 has a substantially concave profile, resulting in a device that is substantially arc- or kidney-shaped. Stated in another way, between the front and back surfaces 16 and 18, a mid or central stem, core or non-linear axis running from the end 21 to the end 23 of the device 1 follows an arc or C-shaped curve 10 as illustrated in FIG. 2. It is foreseen that devices according to the invention may also include both arcuate and linear portions.

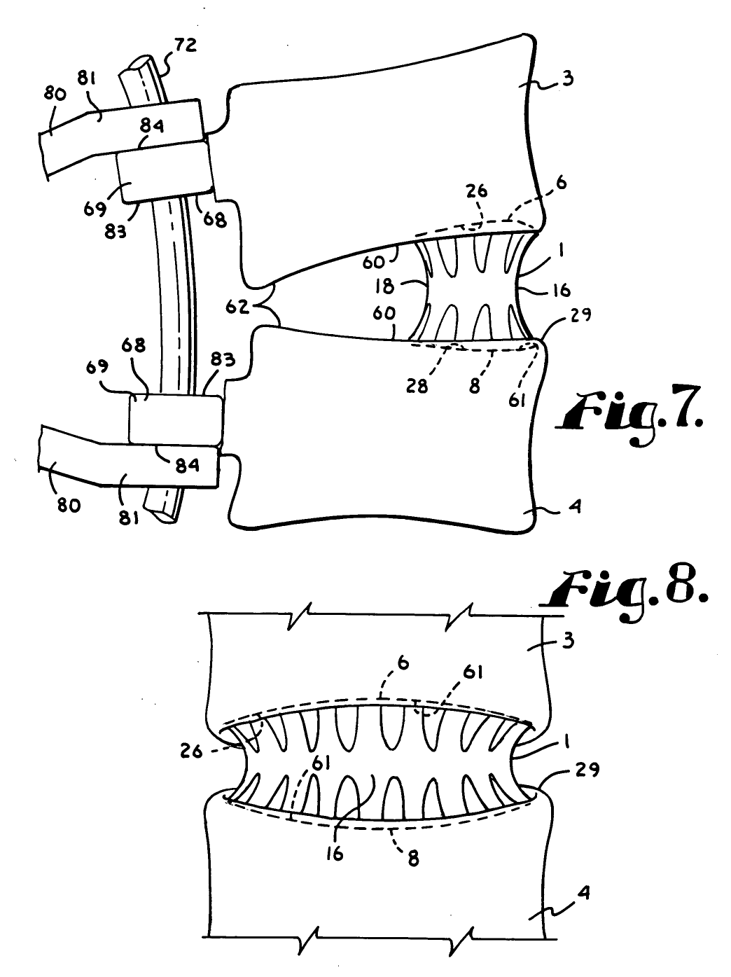

With respect to a vertical dimension running generally between the top surface 6 and the bottom surface 8, the illustrated spacer 1 exhibits a substantially mirror image symmetry on either side of a centrally located horizontal plane 12 as illustrated in FIG. 4. Side or end surfaces 21 and 23 are disposed between and connect the front and back surfaces 16 and 18. The surfaces 21 and 23 have a substantially convex profile when viewed from the top as illustrated in FIG. 2.

The top and bottom surfaces 6 and 8 are smooth and curved and convex when viewed from the side, such as illustrated in FIGS. 4 and 5. The convexity of the abutment or bearing surfaces 6 and 8 is fixed or rigid and conforms to a natural concavity of mutually facing surfaces 26 and 28 of end plates of the adjacent vertebrae 3 and 4. As illustrated in FIG. 9, the arc- or C-shaped device 1 is sized and shaped to generally follow a curvature of an outer edge or perimeter 29 of the vertebral plates 26 and 28, with the front surface 16 closely following or conforming to and slightly spaced from the edge 29 at an anterior location of the vertebrae 3 and 4. It is foreseen that the surfaces 6 and 8 may also include facets or bevels or be radiused at or near edges thereof to promote ease of manipulation during installation.

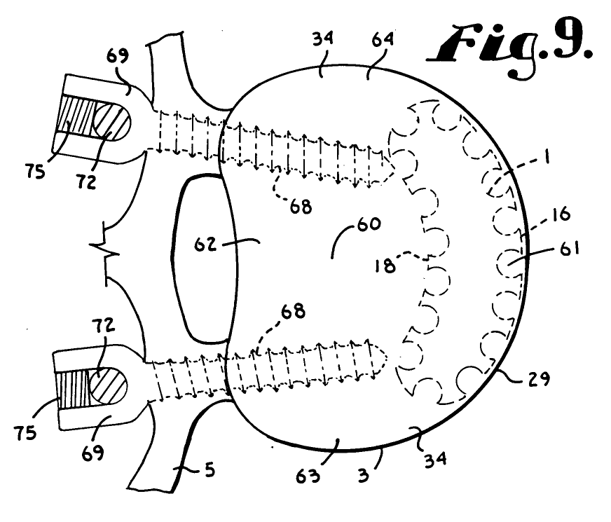

With particular reference to FIGS. 3 and 6, an imaginary convexity plane 30 intersects a peak convexity area of the abutment surfaces 6 and 8 that is in the form of a slightly curved raised portion or ridge 32 located closer to the anterior or front surface 16 than the back surface 18. The substantially anteriorly located area or ridge 32 extends approximately equally on either side of the horizontal plane 12 and corresponds to a concave curvature of the vertebral end plates 26 and 28 at edge regions 34 near the edges 29 of the vertebrae 3 and 4. Also, as illustrated in FIGS. 4-6, because of the overall or end-to-end curvature of the device 1, the device 1 is thus thicker or taller (the distance between abutment surfaces 6 and 8) at a location or plane midway between the end surfaces 21 and 23 than at or near the end surfaces 21 and 23. A reason for the anteriorly shifted or displaced convexity of the device 1 is so that the abutment surfaces 6 and 8 more closely conform to the concavity of the vertebral end plate surfaces 26 and 28, and to produce a correct lordotic alignment of the vertebrae 3 and 4 when implanted as illustrated in FIG. 7. Such shape conformance between the abutment surfaces 6 and 8 and the vertebral surfaces 26 and 28 in combination and cooperating with the overall or end-to-end arcuate shape of the device 1 tends to maximize bearing engagement between the device and the vertebrae 3 and 4 and to minimize possible subsidence of the device 1 into the vertebrae 3 and 4, while providing greater spacing between anterior regions of the vertebrae 3 and 4 than at posterior regions thereof.

The outwardly facing surfaces 16, 18, 21 and 23 are each substantially concave running between the top abutment surface 6 and the bottom abutment surface 8, as shown in FIGS. 1, 3, 4, 5 and 6. The surfaces 16, 18, 21 and 23 form a continuous, curved surface about a periphery of the device 1. The shape of the surfaces 16, 18, 21 and 23 produces upper and lower arcs 36 that provide substantial strength requiring relatively little physical space. In particular, such concavity results in weight reduction in the spacer 1 without appreciably reducing its strength, apparently due to the arched geometry and the fact that such curved surfaces are less likely to create stress risers in the device 1. Additionally, the concave shape leaves more volume between the adjacent vertebrae 3 and 4 to receive fusion promoting bone material. It is foreseen that one or more of the surfaces 16, 18, 21 and 23 could also be faceted rather than curved and still exhibit a substantially concave form. It is also foreseen that some of the surfaces 16, 18, 21 and 23 may be cylindrical or other shape that does not exhibit concavity.

In the illustrated embodiment, a series of side slots or channels 40 are formed on each side surface 16, 18, 21 and 23 of the device 1 so as to pass between the top or superior surface 6 and the bottom or inferior surface 8 while opening laterally outwardly onto a respective surface 16, 18, 21 and 23. The channels 40 run substantially parallel to the plane 30 and have curved innermost ends 46 at either side of the horizontal plane 12 and spaced therefrom. Each channel 40 is preferably about one sixteenth of the length of the device 1 in width measured at the top and bottom surfaces 6 and 8. The channels 40 define feet 48 on either side of each channel 40 that extend toward the top and bottom surfaces 6 and 8 and in a direction outwardly from the central core 10 at the sides 16, 18, 21 and 23 of the device 1, so as to form a vertebrae support matrix while allowing bone growth around the outside of the device through the channels 40. It is foreseen that devices according to the invention may have more, fewer, or no channels 40.

A central body 57 of the device 1 is substantially defined by an intersection of the curved core 10 with the central horizontal plane 12 and extends between superior and inferior surfaces 6 and 8 and also between front and rear surfaces 16 and 18. The central body 57 is substantially solid, being free of pass through bores, windows or the like, so as to form a stable, relatively strong, solid central structure for the device 1. It is foreseen that the central body 57 may include tool gripping apertures.

The device 1 may be formed from any material that has suitable structural properties, is biologically non harmful, and does not promote the growth of pathogens. The material of construction can be biologically active or inactive. For example, various types of metal are suitable as materials of construction. In the illustrated embodiment, the device 1 is formed of a polymer polyester ketone (commonly known as “PEEK”). Composites are also available that satisfy preferred structural and biological requirements. The device can also be made of biologically active or inactive materials, including bone and bone derivatives. The device 1 can be formed by molding, machining, cutting, or the like, or by a combination of such processes to preferably form a single or unitary and substantially rigid structure, preferably with no parts that are moveable relative to other parts thereof.

A method of implanting a spinal fusion spacer device 1 between a pair of adjacent vertebrae 3 and 4 is set forth herein with reference to FIGS. 7-9. It is noted that the upright orientation of the device 1 shown in FIGS. 1-9 is the operational orientation of the spacer device 1 in which the device 1 performs the function of spacing between the vertebrae 3 and 4.

As stated previously, the facing surfaces 26 and 28 of the vertebrae 3 and 4 are somewhat concave in that most of the interior or central regions 60 (FIG. 9) of the surfaces 26 and 28 are spaced farther apart than at the edge regions 34 disposed adjacent to the edges 29. More specifically, with particular reference to FIG. 9, the edge regions 34 that surround each central region 60 may be further described as an anterior edge region 61, a posterior edge region 62 and lateral edge regions 63 and 64. In order to implant a device 1 between the vertebrae 3 and 4, it is preferable to position the vertebrae 3 and 4 far enough apart so that the device 1 can be inserted therebetween and then into alignment as shown in FIGS. 7-9. Insertion is most often by a posterior approach (entry into the posterior edge region 62), but may be from any direction selected by the surgeon. In the present embodiment, the vertebrae 3 and 4 are spread apart during the surgical procedure a sufficient distance that the device 1 can be inserted between the edges 29 and into the edge region 62, followed by manipulation by a tool (not shown) to an eventual use position and location, substantially in the anterior edge region 61 and extending substantially equidistantly toward and somewhat into the lateral edge regions 63 and 64, as shown in FIGS. 7-9.

More specifically, with reference to FIGS. 7 and 9, a pair of open-headed bone screws 68 are threadably implanted into each of the vertebrae 3 and 4. Open heads or receivers 69 of the screws 68 are aligned to receive longitudinal connecting members such as the illustrated spinal fixation rods 72 that run lengthwise along at least a portion of the spine 5 of which the vertebrae 3 and 4 are components. The bone screw heads 69 receive closure structures or plugs 75 which, when tightened, secure the rods 72 within the receivers 69. The receivers 69 and the closure plugs 75 may employ cooperating helical guide and advancement mechanisms to advance the plugs 75 into engagement with the rods 72, as the plugs 75 are rotated into the heads 69. Details of open-headed bone screws 68 and closure structures or plugs 75, which are appropriate for use with the device 1, are found, for example, in applicant's U.S. Pat. No. 6,004,349, which is incorporated herein by reference. The closure structure or plug 75 may be any of a variety of different types of closure structures, including flange form structures with suitable mating structure on the receiver 69, such as illustrated in Applicant's U.S. Pat. No. 6,726,689, which is incorporated herein by reference. Initially the rods 72 are captured only loosely in the receivers 69 by the closure plugs 75, so as to allow movement of the screws 68 along the rods 72 under control of the surgeon.

The vertebrae 3 and 4 are spaced a desired distance by use of a commonly used type scissor tool 80 having spreader arms 81 (shown partially in FIG. 7 in a position for urging the vertebrae toward one another subsequent to installation of the device 1). The plugs 75 may be lightly tightened in the bone screws 68. At such point in the procedure, the tool 80 is used to press against facing surfaces 83 of the bone screw receivers 69, to press the bone screws 68 away from one another, that in turn moves the vertebrae 3 and 4 away from one another, providing a desired intervertebral distance that enables insertion of the spacer device 1 between the vertebrae 3 and 4 at the posterior edge region 62. The spacer device 1 is inserted between the spread vertebrae 3 and 4 with an insertion tool (not shown) to the substantially anterior position in the region 61 as shown in FIGS. 7 and 9 with the front surface 16 disposed near and substantially evenly spaced from the curved anterior edges 29 of the vertebrae 3 and 4.

As indicated previously herein, the plugs 75 are lightly tightened during the implantation of the device 1. Then, with reference to FIG. 7, the plugs 75 are loosened and the arms 81 of the tool 80 are placed on opposite surfaces 84 of the receivers 69 that face away from one another to perform a pressing procedure that urges the screws 68 of adjacent vertebrae 3 and 4 toward each other, so that the posterior ends of the vertebrae 3 and 4 become more closely spaced to allow the inner surfaces 26 and 28 respectively thereof to snugly engage the superior and inferior abutment surfaces 6 and 8 of the device 1, preferably in a clamping relationship, as shown in FIGS. 7 and 8. Such clamping secures the device 1 in the desired position selected therefor between the vertebrae 3 and 4. The orientation of the device 1 with respect to the vertebrae 3 and 4 is adjusted, if necessary, prior to final tightening of the plugs 75 to lock the relative position between a respective rod 72 and the screws 68. Prior to insertion or after insertion in the bone screws 68, the rods 72 may be bent somewhat to achieve a desired angular or lordotic relationship between the vertebrae 3 and 4. A single device 1 when used in conjunction with a pair of bone screws 68 in each vertebra 3 and 4 forms a solid multiple point of support structure so as to stabilize the vertebrae 3 and 4 with respect to each other. In the illustrated embodiment there is a stable three point support (two bone screws 68 and the device 1) provided for each vertebra 3 and 4 relative to the adjacent vertebra 3 or 4.

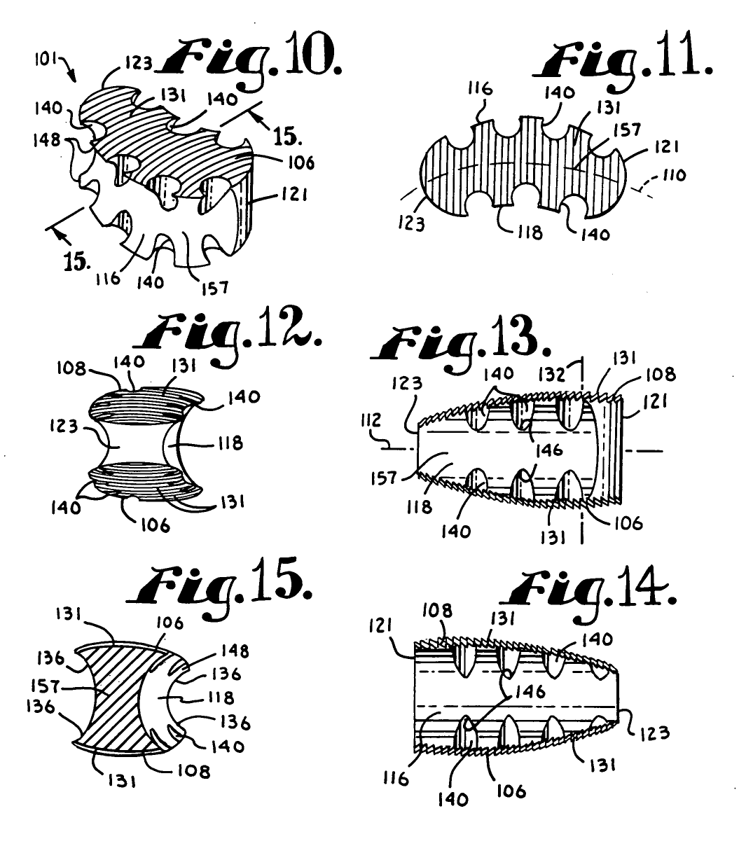

With reference to FIGS. 10-19, an alternative or modified embodiment, generally 101, of a spacer device according to the invention is similar to, but smaller than the device 1, and sized and shaped for use in cooperation with a second, inverted and oppositely facing identical device 102 as illustrated in FIG. 18. The paired devices 101 and 102 are used to maintain proper spacing between a pair of adjacent vertebrae 103 and 104 of a human spine 105 as a replacement for the intervertebral disc and to promote fusion between the vertebrae 103 and 104.

The device 101 has a superior or first vertebra abutment surface 106 and an inferior or second vertebra abutment surface 108. The device 101 further includes opposed first and second, or outer and inner, side surfaces 116 and 118, respectively, the surfaces 116 and 118 substantially facing away from one another. In use, the first or outer surface 116 faces laterally outwardly from the vertebrae 103 and 104 while the second or inner surface 118 substantially faces toward an interior of the vertebrae 103 and 104 and also toward an inward surface 118′ of the paired device 102. When viewed from the top or bottom, as illustrated in FIG. 11, the outward surface 116 has a substantially convex profile while the inner or inwardly facing surface 118 has a substantially concave profile, resulting in a device that is substantially arc- cashew- or kidney-shaped. Stated in another way, between the first or outer surface 116 and the second or inner surface 118, a central stem, axis or core of the device 101 is non-linear, following a generally arc- or C-shaped curve 110 as illustrated in FIG. 11.

With respect to a vertical dimension running generally between the abutment surface 106 and the abutment surface 108, the illustrated spacer 101 exhibits a substantially mirror image symmetry on either side of a centrally located horizontal plane 112 as illustrated in FIG. 13. Front or anterior and rear or posterior end surfaces 121 and 123, respectively, are disposed between and connect the front and back surfaces 116 and 118. The surfaces 121 and 123 each have a substantially convex profile when viewed from the top or bottom as illustrated in FIG. 11. The surfaces 121 and 123 are cylindrical and substantially perpendicular to the plane 112. The surfaces 116 and 118 are concave as will be discussed in greater detail below.

The abutment surfaces 106 and 108 are curved and convex when viewed from the side, such as illustrated in FIGS. 12-15. The convexity of the abutment or bearing surfaces 106 and 108 is fixed or rigid and conforms to a natural concavity of mutually facing surfaces 126 and 128 of end plates of the adjacent vertebrae 103 and 104. As illustrated in FIG. 18, the arc- or C-shaped devices 101 and 102 are sized and shaped to generally follow a curvature of an outer edge or perimeter 129 of the vertebral plates 126 and 128, with the first or laterally facing surface 116 closely following and slightly spaced from the edge 129 at a side or lateral location along the vertebrae 103 and 104.

The surfaces 106 and 108 further include ridges, ribs or teeth 131 running between the opposed sides 116 and 118. The ribbed or toothed surfaces 131 provide gripping engagement with the vertebral surfaces 126 and 128 to aid in holding the spacer 101 in place between the vertebrae 103 and 104. Furthermore, such ridges or teeth 131 aid in keeping the spacer 101 in a desired orientation between the adjacent vertebrae 103 and 104 until fusion between the vertebrae 103 and 104 occurs. As illustrated in FIGS. 18 and 19, when implanted, points of the teeth 131 are directed toward the anterior region of the vertebrae, aiding in keeping the device in a desired location both during and subsequent to bone screw tightening as will be described more fully below. It is foreseen that the abutment surfaces 106 and 108 may also be beveled or radiused at or near edges thereof to promote ease of manipulation during installation.

With particular reference to FIG. 13, an imaginary convexity plane 132 intersects a peak convexity area or ridge of the abutment surfaces 106 and 108 located near the end surface 121. The substantially laterally located peak convexity extends approximately equally on either side of the horizontal plane 112 and thus is where the device 101 exhibits a maximum vertical thickness. A reason for the anteriorly shifted or displaced convexity of the device 101 is so that the abutment surfaces 106 and 108 not only conform to the concavity of the vertebral end plate surfaces 126 and 128, but also produce a correct lordotic alignment of the vertebrae 103 and 104 when implanted as illustrated in FIGS. 16-19. As illustrated in FIG. 12, the peak convexity area also bears slightly closer to the inner side surface 118 than the outer or laterally facing side surface 116. Such shape conformance between the abutment surfaces 106 and 108 and the vertebral surfaces 126 and 128 tends to maximize bearing engagement between the vertebrae 103 and 104 and, in combination with the end-to-end curvate nature of the device 101, tends to minimize possible subsidence of the device 101 into the vertebrae 103 and 104, while providing greater spacing between anterior ends of the vertebrae 103 and 104 than at posterior ends thereof.

The outwardly facing surfaces 116 and 118 are both substantially concave running between the abutment surface 106 and the abutment surface 108, as shown in FIGS. 10 and 12-14. It is foreseen that the surfaces 116 and 118 and also the surfaces 121 and 123 could be faceted. It is foreseen that the surfaces 121 and 123 could also be concave. The shape of the surfaces 116 and 118 produces upper and lower arcs 136 that provide substantial strength requiring relatively little physical space. In particular, such concavity results in weight reduction in the spacer 101 without appreciably reducing its strength, apparently due to the arched geometry and the fact that such curved surfaces are less likely to create stress risers in the device 101. Addtionally, the concave shape leaves more volume between the adjacent vertebrae 103 and 104 to receive fusion promoting bone material.

In the illustrated embodiment a series of side slots or channels 140 are formed on each surface 116 and 118 of the device 101 so as to pass between the abutment surface 106 and the abutment surface 108 while opening laterally outwardly onto a respective outer side surface 116 or inner side surface 118. The channels 140 run substantially parallel to the plane 132 and have curved innermost ends 146 at either side of the horizontal plane 112 and spaced therefrom. Each channel 140 is preferably about one eighth of the length of the device 101 in width measured at the top and bottom surfaces 106 and 108. The channels 140 further define feet 148 on either side of each channel 140 that extend toward the abutment surfaces 106 and 108 and in a direction outwardly from the central core 110 at the sides 116 and 118 of the device 101, so as to form a vertebral support matrix while allowing bone growth around the outside of the device through the channels 140.

A central body 157 of the device 101 is substantially defined by an intersection of the curved core 110 with the central horizontal plane 112 and extends between the abutment surfaces 106 and 108 and also between first and second side surfaces 116 and 118. The central body 157 is substantially solid, being free of pass through bores, windows or the like, so as to form a stable, relatively strong, solid central structure for the device 101. It is foreseen that the central body 157 may include tool gripping apertures.

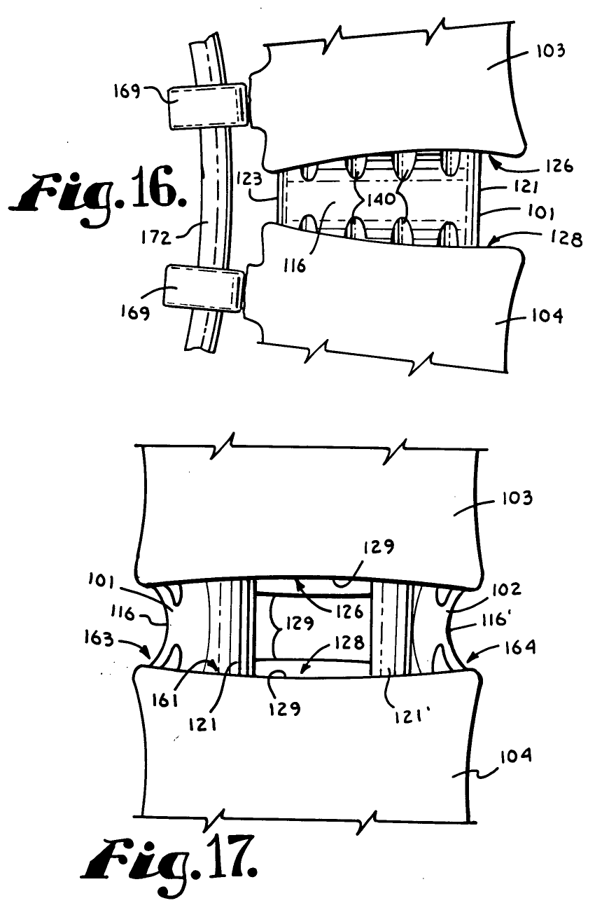

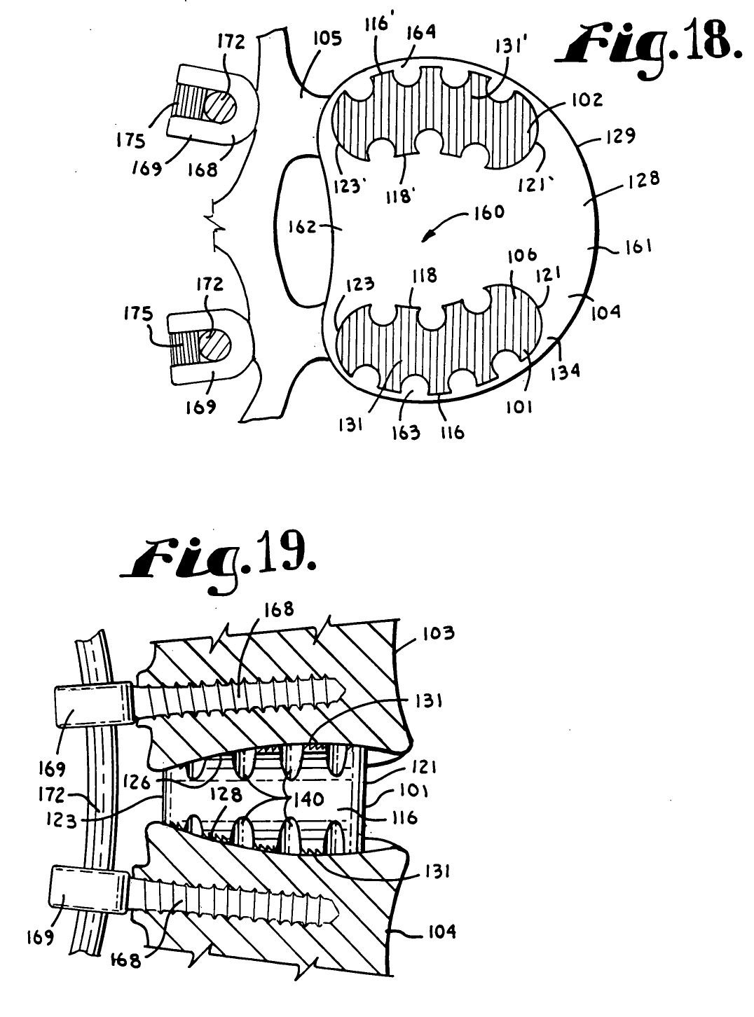

With reference to FIGS. 16-19, the device 101 is implanted in cooperation with an identical device 102, with the device 102 being oriented inverted and opposite the device 101, as particularly illustrated in FIGS. 17 and 18. Thus, the device 102 includes abutment surfaces 106′ and 108′ identical to the surfaces 106 and 108, outward and inward surfaces 116′ and 118′ identical to surfaces 116 and 118, end surfaces 121′ and 123′ identical to surfaces 121 and 123, as well as all the other features previously discussed herein with respect to the device 101. The devices 101 and 102 are each aligned in spaced relation to outer edges 129 at lateral regions or sides of the vertebral plates 126 and 128, with the ends 123 and 123′ located in posterior regions of the vertebrae 103 and 104.

The implantation procedure is similar to that described previously herein with respect to the device 1, with the exception that two devices 101 and 102 are each inserted between the vertebrae 103 and 104 to opposed lateral positions as best illustrated in FIGS. 17 and 18. Unlike the device 1, the devices 101 and 102 can be inserted into the edge regions 161 in a laterally laid-over or tipped-over orientation and then rotated ninety degrees to the upright orientation shown in FIGS. 16-19.

The facing surfaces 126 and 128 of the vertebrae 103 and 104 are somewhat concave in that most of the interior or central region 160 of the surfaces 126 and 128 are spaced farther apart than at the edge regions 134 disposed adjacent to the edges 129. More specifically, with particular reference to FIG. 18, the edge regions 134 that surround each central region 160 may be further described as an anterior edge region 161, a posterior edge region 162 and lateral edge regions 163 and 164. Similar to what has been described herein with respect to the device 1, in order to implant devices 101 and 102 between the vertebrae 103 and 104, the vertebrae 103 and 104 are spread apart and the devices 101 and 102 are inserted, most often into the posterior edge region 162, but may be from any direction selected by the surgeon. The device 101 is then manipulated into the lateral region 163 and the device 102 is manipulated into the lateral region 164, with the taller end surfaces 121 and 121′ extending into the anterior region 161 and the,shorter ends 123 and 123′ located in the posterior region 162. The surfaces 116 and 116′ are both aligned with and face toward the vertebral edges or peripheries 129, with the surfaces 118 and 118′ facing the central region 160 and generally facing one another.

With reference to FIGS. 18 and 19, similar to the discussion herein with respect to the implantation of the device 1, a pair of open-headed bone screws 168 previously implanted into each of the vertebrae 103 and 104 include open heads or receivers 169 aligned to receive longitudinal connecting members such as the illustrated spinal fixation rods 172 that run lengthwise along at least a portion of the spine 105 of which the vertebrae 103 and 104 are components. The plugs 175 may be lightly tightened in the bone screws 168 during implantation of the devices 101 and 102 when the vertebrae are spread apart with a scissor tool (not shown). Once the devices 101 and 102 are positioned in the desired locations illustrated in FIG. 18, the scissor tool may be used to press the vertebrae 103 and 104 toward one another. As the vertebrae are pressed toward one another, primarily at the posterior regions 162 any anterior movement or forward urging of the spacers 101 and 102 that such pressing might cause is prohibited by the teeth 131 penetrating the surfaces 126 and 128 of the respective vertebrae 103 and 104.

When a desired degree of engagement between the vertebrae 103 and 104 and the two devices 101 and 102, along with the desired orientation of the devices 101 and 102 relative to the vertebrae 3 and 4 is ultimately achieved, the closure plugs 175 are advanced into secure engagement with rods 172 in a substantially permanent relation. Such an embodiment provides four points of support for each vertebrae 103 and 104 relative to the adjacent vertebrae 103 or 104.

Any voids between the vertebrae 3 and 4 or vertebrae 103 and 104 and respective adjacent device 1 or paired devices 101 and 102 are preferably packed with bone material that over time will promote fusion between such vertebrae, so that the adjacent vertebrae will eventually be locked in the spacing and orientations established by the spacer device 1 or devices 101 and 102 as well as the cooperating rods.

The devices 1 and 101 include no moving or adjustable parts and may be manufactured from biologically inactive materials or from biologically active materials that are compatible with implantation. The devices 1 and 101 formed of biologically inactive materials are chemically and biologically essentially inert in their implanted environments. Fusion of the vertebrae occurs around the devices 1 and 101 and through respective side channels thereof. However, the devices 1 and 101 remain intact after implantation.

The biologically inactive materials used for the devices 1 and 101 can be divided into metallic materials and non-metallic materials. Metallic biologically inactive materials may include certain stainless steel alloys, titanium, and tantalum and other alloys and materials which are structurally, chemically, and biologically appropriate. Non-metallic biologically inactive materials for the devices 1 and 101 can include certain plastics or polymers, organic and inorganic resins, composites, and ceramics, especially polyester ketone or the polymer commonly referred to as “PEEK”. The polymers are preferably non-porous. The composites may include carbon fiber reinforced materials. Appropriate ceramics are preferably porous and can be of an “open scaffold” type which allow bone fusion growth through the ceramic material itself.

The devices 1 and 101 can also be formed from biologically active materials which are normally biologically substituted for, absorbed, or otherwise replaced as bone fusion of the vertebrae proceeds. The biologically active materials can be either bone-based or non-bone-based. The term bone-based material is used herein to refer to a material which is made from actual bones, bone derivatives, or materials which are chemically bone-like. Bones are typically formed mostly (about 85 percent) of tri-basic calcium phosphate which, in living bone, is called hydroxy-apatite or simply calcium phosphate. In general, the bone is formed by cutting, machining or the like or bone derived material is ground, mixed with a suitable resin or other binder, and cast, molded or machined to shape. Further machining or other mechanical forming may be performed in final shaping of formed implant spacers. The source of bone for such material is possibly a harvest from another part of the patient who will receive the implant or from cadaver bone or allograft. Other sources may include non-human bone.

Biologically active, non-bone-based materials appropriate for use in the devices 1 and 101 include corals, certain resins and similar materials. The principal constituent of coral is calcium carbonate in a porous form which allows bone fusion growth through the resulting spacer. The devices 1 and 101 can be formed of coral by machining or carving processes. The coral material is normally replaced over time by biological processes in the body, as the spinal fusion process occurs.

It is to be understood that while certain forms of the present invention have been illustrated and described herein, it is not to be limited to the specific forms or arrangement of parts described and shown.

What is claimed and desired to be secured by Letters Patent is as follows:

1. An interbody device for placement between a pair of opposing vertebrae; said device comprising:
(a) opposed abutment surfaces sized and shaped for engaging adjacent spaced vertebrae;
(b) first and second opposed sides, each side disposed between the abutment surfaces with at least the first side having a substantially concave surface; and
(c) a body disposed between the first and second sides and between the opposed abutment surfaces, the body being substantially non-linear in a direction running along and between the first and second sides.

2. The device of claim 1 wherein the first and second sides each have a substantially concave surface.

3. The device of claim 1 wherein the first side has a substantially convex profile and the second side has a substantially concave profile.

4. The device of claim 3 wherein the convex profile of the first side is sized and shaped to conform to and be placed in spaced relation to a peripheral edge of a cooperating vertebra.

5. The device of claim 1 further comprising at least one and up to a plurality of channels extending between the abutment surfaces and opening laterally onto the first side.

6. The device of claim 1 further comprising at least one and up to a plurality of channels extending between the abutment surfaces and opening laterally onto the second side.

7. The device of claim 1 wherein each of the abutment surfaces are convex.

8. The device of claim 7 wherein the abutment surfaces are asymmetrically convex, defining a maximum thickness of the device located near the first side.

9. The device of claim 7 wherein the abutment surfaces are asymmetrically convex, defining a maximum thickness of the device positioned for placement near an anterior edge of the vertebrae.

10. The device of claim 1 constructed from a single, solid, unitary structure.

11. The device of claim 1 wherein the abutment surfaces have teeth.

12. The device of claim 11 wherein the teeth are in the form of ridges extending between the first and second sides and are angled with points thereof directed toward an anterior of the vertebrae when implanted.

13. The device of claim 1 wherein the device is a first spacer and further comprising a second spacer, the first and second spacers being substantially identical, the first side of each of the spacers sized and shaped to be positioned near a lateral edge of a vertebra, with the first and second spacers positioned facing one another and the second spacer being in an inverted position.

14. An interbody device for placement between a pair of opposing vertebrae; said device comprising:
(a) a substantially arcuate body having opposed abutment surfaces sized and shaped for engaging adjacent spaced vertebrae; and
(b) first and second opposed sides running between the abutment surfaces, the first side having a convex profile, the second side having a concave profile, and at least the first side having a substantially concave surface.

15. The device of claim 14 wherein the convex profile of the first side is sized and shaped to conform to a curvature of an anterior edge of a cooperating vertebra.

16. The device of claim 14 wherein the convex profile of the first side is sized and shaped to conform to a curvature of a lateral edge of a cooperating vertebra.

17. The device of claim 14 wherein the first side and the second side each have a substantially concave surface.

18. The device of claim 14 further comprising at least one and up to a plurality of channels extending between the abutment surfaces and opening laterally onto the first side.

19. The device of claim 14 further comprising at least one and up to a plurality of channels extending between the abutment surfaces and opening laterally onto the second side.

20. The device of claim 14 further comprising first and second ends and wherein each of the first side, the second side, the first end and the second end have a concave surface.

21. The device of claim 20 wherein the concave surfaces of the first and second sides cooperate with the concave surfaces of the first and second ends forming a continuous concave surface about a periphery of the device.

22. The device of claim 14 further comprising first and second ends and wherein the first and second sides have a concave surface and the first and second ends have a cylindrical surface.

23. The device of claim 22 wherein a first thickness of the device measured between the abutment surfaces near the first end is greater than a second thickness measure between the abutment surfaces near the second end.

24. The device of claim 14 wherein the body has a solid arcuate core.

25. The device of claim 14 wherein each of the abutment surfaces are convex.

26. The device of claim 25 wherein the abutment surfaces are asymmetrically convex, defining a maximum thickness of the device located near the first side.

27. The device of claim 25 wherein the abutment surfaces are asymmetrically convex, defining a maximum thickness of the device positioned for placement near an anterior edge of the vertebrae.

28. The device of claim 14 wherein the abutment surfaces have teeth.

29. The device of claim 28 wherein the teeth are in the form of ridges extending between the first and second sides and are angled with points thereof directed toward an anterior of the vertebrae when implanted.

30. The device of claim 14 wherein the device is a first spacer and further comprising a second spacer, the first and second spacers being substantially identical, the first side of each of the spacers sized and shaped to be positioned near a lateral edge of a vertebra, with the first and second spacers positioned facing one another and the second spacer being in an inverted position.

31. In a spinal fusion interbody spacer for placement between adjacent vertebrae, the spacer having opposed abutment surfaces for engaging the adjacent vertebrae, and substantially oppositely facing first and second surfaces extending between the abutment surfaces, the improvement wherein the abutment surfaces and the first and second surfaces define an elongate body substantially non-linear along a length thereof running in spaced relation to the first and second surfaces, with at least one of the first and second surfaces being substantially concave.

32. The improvement of claim 31 wherein the abutment surfaces are each convex.

33. The improvement of claim 31 wherein the first and second surfaces are each concave.

34. The improvement of claim 31 wherein the first surface has a convex profile and the second surface has a concave profile.

35. The improvement of claim 31 wherein the first surface is sized and shaped to conform to a curved shape of a periphery of a vertebra when positioned in spaced relation thereto.

36. In a spinal fusion interbody spacer for placement between adjacent vertebrae, the spacer having opposed abutment surfaces for engaging the adjacent vertebrae and substantially oppositely facing anterior and posterior surfaces, the improvement wherein the abutment surfaces and the anterior and posterior surfaces substantially define an arcuate body of the spacer and at least one of the anterior and posterior surfaces is substantially concave.

37. The improvement of claim 36 wherein the abutment surfaces are each convex.

38. The improvement of claim 36 wherein each of the anterior and posterior surfaces are concave.

39. The improvement of claim 36 wherein the anterior surface has a convex profile and the posterior surface has a concave profile.
